(19) United States
(12) Patent Application Publication
(10) Pub. No.: US 2025/0205225 A1
(43) Pub. Date: Jun. 26, 2025

(54) METHODS OF POTENTIATING TEMOZOLOMIDE ACTIVITY AGAINST GLIOBLASTOMA CELLS

(71) Applicant: University of Cincinnati, Cincinnati, OH (US)

(72) Inventors: Pankaj B. Desai, Cincinnati, OH (US); Aniruddha S. Karve, Cincinnati, OH (US); Janki Mihir Desai, Lawrenceburg, IN (US); Timothy Phoenix, Blue Ash, OH (US); Nimita Dave, Roslindale, MA (US); Gary Gudelsky, Cincinnati, OH (US)

(73) Assignee: University of Cincinnati, Cincinnati, OH (US)

(21) Appl. No.: 18/848,646

(22) PCT Filed: Mar. 23, 2023
(86) PCT No.: PCT/US2023/016029

§ 371 Date: Sep. 19, 2024

Related U.S. Application Data
(60) Provisional application No. 63/323,053, filed on Mar. 23, 2022.

(51) Int. Cl. A61K 31/495; A61K 31/175; A61K 31/4196; A61K 39/395; A61P 35/00

(52) U.S. Cl. CPC A61K 31/495; A61K 31/175; A61K 31/4196; A61K 39/3955; A61P 35/00

(57) ABSTRACT

Provided herein is a method of potentiating a cytotoxic effect of temozolomide against glioblastoma cells in a subject in need thereof, the method including administering to the subject a combination of: a therapeutically effective amount of temozolomide; and a non-cytotoxic amount of letrozole, wherein the letrozole potentiates the cytotoxic effect of the temozolomide against the glioblastoma cells. Also provided is a method of restoring sensitivity of temozolomide-resistant glioblastoma cells to temozolomide, including contacting the glioblastoma cells with a non-cytotoxic amount of letrozole and method of treating glioblastoma.

### CROSS REFERENCE TO RELATED APPLICATION

This application claims priority to U.S. Provisional Application Ser. No. 63/323,053, filed Mar. 23, 2022, the entire contents of which are incorporated herein by reference.

### TECHNICAL FIELD

The present disclosure relates to the field of cancer therapy. More specifically, the present disclosure relates to methods of potentiating the activity of temozolomide against glioblastoma multiforme cells by administering a non-cytotoxic amount of letrozole.

### BACKGROUND

Glioblastoma multiforme (GBM) is a highly aggressive and lethal cancer of the central nervous system (CNS). The current standard therapy for GBM patients comprises surgical resection followed by concomitant fractionated radiotherapy and chemotherapy with the DNA methylating agent temozolomide (TMZ), 7 days a week for 6 weeks at a dose of 75 mg/m². This is followed by six cycles of adjuvant temozolomide administration at a dose of 150-200 mg/m², with each 28-day cycle consisting of 5 days of temozolomide administration. Unfortunately, this multimodal approach is not curative and only extends the median survival from 4 months to approximately 15 months. Even initially TMZ-sensitive tumors acquire drug resistance and recur in almost all patients. Temozolomide is an alkylating agent prodrug, delivering a methyl group to the N⁷ or O⁶ position of guanine residues of DNA, with the primary lesion formed being O⁶-methylguanine (O⁶-MeG). The methylation leads to DNA strand breaks and triggers apoptotic tumor cell death. The mechanisms of resistance to temozolomide are complex and may result from selection of pre-existing resistant clones or genetic and epigenetic alterations of neoplastic cells. One of the primary mechanisms of resistance entails the overexpression of the repair protein O⁶-methlyguanine-DNA-methyltransferase (MGMT), which protects the cellular genome by removing the O⁶-alkylguanine DNA adduct. Hypermethylation of the MGMT promoter results in decreased expression of the MGMT protein and is associated with increased sensitivity to temozolomide, whereas a reduction in MGMT promoter methylation leads to the development of resistance to DNA alkylating agents and tumor recurrence. In the majority of cases, recurrent GBMs are resistant to radiation and chemotherapy at the currently approved standard dosage. In addition to this clinical limitation, temozolomide use in GBM patients is associated with serious adverse effects. These include commonly observed gastrointestinal (nausea, vomiting and diarrhea), CNS (convulsions and headaches), and hematologic (neutropenia and thrombocytopenia) toxicities.

A need exists for improved strategies to enhance temozolomide activity against GBM cells and overcome the development of resistance to improve outcomes and quality of life for diagnosed patients and their families.

### SUMMARY

Accordingly, provided herein are methods of potentiating the apoptosis-inducing effects of temozolomide against GBM cells by co-administering a sub-cytotoxic amount of letrozole. Advantageously, it has been found that letrozole and temozolomide work synergistically in combination to potentiate temozolomide in TMZ-sensitive, intermediately sensitive, and resistant GBM cells.

In one embodiment, a method of potentiating the cytotoxic effect of temozolomide against glioblastoma cells in a subject in need thereof is provided, the method comprising administering to the subject a combination of: a therapeutically effective amount of temozolomide; and a non-cytotoxic amount of letrozole, wherein the letrozole potentiates the cytotoxic effect of the temozolomide against the glioblastoma cells.

In another embodiment, a method of treating glioblastoma in a subject in need thereof is provided, the method comprising administering to the subject a combination of: a therapeutically effective amount of temozolomide; and a non-cytotoxic amount of letrozole.

In another embodiment, a method of restoring the sensitivity of temozolomide-resistant glioblastoma cells to temozolomide is provided, the method comprising contacting the glioblastoma cells with a non-cytotoxic amount of letrozole.

In another embodiment, a combination of a therapeutically effective amount of temozolomide and a non-cytotoxic amount of letrozole is provided for use in a method of treating glioblastoma in a subject in need thereof.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1A shows the effects of drug treatment (LTZ, TMZ, LTZ (40 nM)+TMZ, and LTZ (40 nM)+TMZ+E2) on viability of patient-derived TMZ-sensitive G76 glioblastoma cells.

FIG. 1B shows the effects of drug treatment (LTZ, TMZ, LTZ (40 nM)+TMZ, and LTZ (40 nM)+TMZ+E2) on viability of patient-derived TMZ-intermediately sensitive BT-142 glioblastoma cells.

FIG. 1C shows the effects of drug treatment (LTZ, TMZ, LTZ (40 nM)+TMZ, and LTZ (40 nM)+TMZ+E2) on viability of patient-derived TMZ-resistant G43 glioblastoma cells.

FIG. 1D shows the effects of drug treatment (LTZ, TMZ, LTZ (40 nM)+TMZ, and LTZ (40 nM)+TMZ+E2) on viability of patient-derived TMZ-resistant G75 glioblastoma cells.

FIG. 2A shows microscopic analysis of the cytotoxic effects of LTZ and TMZ on neurosphere growth of BT-142 glioblastoma cells. Cells (1.4×10³/well) were treated with solvent control (0.1% DMSO) and the indicated concentration of LTZ and TMZ (n=3 per treatment group) with or without the addition of exogenous estradiol (250 pg/mL) for 72 hours. Representative microscopic images are shown for BT-142-gfp-luc cells treated with vehicle, LTZ (40 nM), TMZ (1 μM), TMZ (1 μM)+LTZ (40 nM) and TMZ (1 μM)+LTZ (40 nM)+E2. Scale displayed on each image=0.2 mm.

FIG. 2B shows microscopic analysis of the cytotoxic effects of LTZ and TMZ on neurosphere growth of G43 glioblastoma cells. Cells (1.4×10³/well) were treated with solvent control (0.1% DMSO) and the indicated concentration of LTZ and TMZ (n=3 per treatment group) with or without the addition of exogenous estradiol (250 pg/mL) for 72 hours. Representative microscopic images are shown for G43 cells treated with vehicle, LTZ (40 nM), TMZ (1 μM), TMZ (1 μM)+LTZ (40 nM) and TMZ (1 μM)+LTZ (40 nM)+E2. Scale displayed on each image=0.2 mm.

FIG. 2C is a plot of the relative diameters of the treated BT-142-gfp-luc cells shown in FIG. 2A (data: mean±SD).

FIG. 2D is a plot of the relative diameters of the treated G43 cells shown in FIG. 2B (data: mean±SD).

FIG. 3A is a combination index (CI) plot showing the interaction of TMZ+LTZ and the effect of estradiol addition (250 pg/ml). CI plots for cell viability are shown for G76, G75, BT-142, and G43 cells, each with or without E2.

FIG. 3B is a combination index (CI) plot showing the interaction of TMZ+LTZ and the effect of estradiol addition (250 pg/ml). CI plots for neurosphere size measurement are shown for BT-142 and G43 cells, each with or without E2.

FIG. 4A depicts flow cytometry dot plots exhibiting induction of γH2A.X in G76 cells treated with control, LTZ (40 nM), TMZ (0.1 μM), and LTZ (40 nM)+TMZ (0.1 μM) (with and without E2) for 5 h. The EC₅₀ for DNA damage for each treatment was predicted with Phoenix® WinNonLin v8.3 using a standard Emax model, described in FIG. 9. (data: mean SD).

FIG. 4B depicts flow cytometry dot plots exhibiting induction of γH2A.X in B-142 cells treated with control, LTZ (40 nM), TMZ (0.1 μM), and LTZ (40 nM)+TMZ (0.1 μM) (with and without E2) for 5 h. The EC₅₀ for DNA damage for each treatment was predicted with Phoenix® WinNonLin v8.3 using a standard Emax model, described in FIG. 9. (data: mean SD).

FIG. 4C depicts flow cytometry dot plots exhibiting induction of γH2A.X in G43 cells treated with control, LTZ (40 nM), TMZ (0.1 μM), and LTZ (40 nM)+TMZ (0.1 μM) (with and without E2) for 5 h. The EC₅₀ for DNA damage for each treatment was predicted with Phoenix® WinNonLin v8.3 using a standard Emax model, described in FIG. 9. (data: mean SD).

FIG. 4D depicts flow cytometry dot plots exhibiting induction of γH2A.X in G75 cells treated with control, LTZ (40 nM), TMZ (0.1 μM), and LTZ (40 nM)+TMZ (0.1 μM) (with and without E2) for 5 h. The EC₅₀ for DNA damage for each treatment was predicted with Phoenix® WinNonLin v8.3 using a standard Emax model, described in FIG. 9. (data: mean SD).

FIG. 5A is a plot showing the induction of caspase 3/7 activity as a marker for apoptosis in G76 cells treated with LTZ, TMZ, LTZ (40 nM)+TMZ, and LTZ (40 nM)+TMZ+E2 (250 pg/mL) for 24 h (n=3 per treatment group). Caspase activity was determined using the Caspase-Glo 3/7 assay kit (normalized with Promega CellTiter-Glo® Luminescent Cell Viability assay). The EC₅₀ for caspase induction for each treatment was predicted with Phoenix® WinNonLin v8.3 using a standard Emax model, described in FIG. 9.

FIG. 5B is a plot showing the induction of caspase 3/7 activity as a marker for apoptosis in BT142 cells treated with LTZ, TMZ, LTZ (40 nM)+TMZ, and LTZ (40 nM)+TMZ+E2 (250 pg/mL) for 24 h (n=3 per treatment group). Caspase activity was determined using the Caspase-Glo 3/7 assay kit (normalized with Promega CellTiter-Glo® Luminescent Cell Viability assay). The EC₅₀ for caspase induction for each treatment was predicted with Phoenix® WinNonLin v8.3 using a standard Emax model, described in FIG. 9.

FIG. 5C is a plot showing the induction of caspase 3/7 activity as a marker for apoptosis in G43 cells treated with LTZ, TMZ, LTZ (40 nM)+TMZ, and LTZ (40 nM)+TMZ+E2 (250 pg/mL) for 24 h (n=3 per treatment group). Caspase activity was determined using the Caspase-Glo 3/7 assay kit (normalized with Promega CellTiter-Glo® Luminescent Cell Viability assay). The EC₅₀ for caspase induction for each treatment was predicted with Phoenix® WinNonLin v8.3 using a standard Emax model, described in FIG. 9.

FIG. 5D is a plot showing the induction of caspase 3/7 activity as a marker for apoptosis in G75 cells treated with LTZ, TMZ, LTZ (40 nM)+TMZ, and LTZ (40 nM)+TMZ+E2 (250 pg/mL) for 24 h (n=3 per treatment group). Caspase activity was determined using the Caspase-Glo 3/7 assay kit (normalized with Promega CellTiter-Glo® Luminescent Cell Viability assay). The EC₅₀ for caspase induction for each treatment was predicted with Phoenix® WinNonLin v8.3 using a standard Emax model, described in FIG. 9.

FIG. 6A is a Western blot depicting aromatase expression across the patient-derived GBM cell lines G43, BT-142, G75, and G76.

FIG. 6B is a Western blot depicting aromatase expression across the patient-derived GBM cell lines G43, BT-142, G75, and G76. The depicted rows are selected from FIG. 6A.

FIG. 7 shows the time course of γH2A.X induction in G75 cells after treatment with vehicle, LTZ (0.04 μM), TMZ (0.1 μM), LTZ (0.04 μM)+TMZ (0.1 μM), and LTZ (0.04 μM)+TMZ (0.1 μM)+E2. Based on this analysis, 5 h after treatment was chosen as the time point for analysis of γH2A.X.

FIG. 8 is a table showing IC₅₀ and CI values for LTZ, TMZ, and LTZ (40 nM)+TMZ (±E2) against patient-derived GBM lines obtained from cell viability assays and neurosphere growth inhibition.

FIG. 9 is a table showing EC₅₀ and CI values for DNA damage (γH2A.X) and caspase induction in GBM cells treated with LTZ, TMZ, and LTZ (40 nM)+TMZ (±E2).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an,” and “the” include plural references unless the content clearly dictates otherwise.

Letrozole (4,4′-((1H-1,2,4-triazol-1-yl)methylene)dibenzonitrile) is a non-steroidal aromatase inhibitor having the following chemical structure:

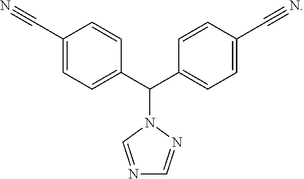 [SMILES: N#Cc1ccc(C(c2ccc(C#N)cc2)n2cncn2)cc1]

Temozolomide (4-methyl-5-oxo-2,3,4,6,8-pentazabicyclo[4.3.0]nona-2,7,9-triene-9-carboxamide) is an alkylating agent having the following chemical structure:

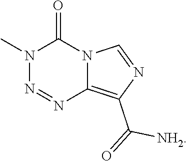

The term “potentiate,” as used herein, refers to increasing the potency or effectiveness of a drug or treatment. Advantageously, the inventors have shown that sub-cytotoxic levels of letrozole potentiate the apoptosis-inducing effect of temozolomide against glioblastoma cells, including TMZ-sensitive, TMZ-intermediately sensitive, and TMZ-resistant glioblastoma cells.

As used herein, the terms “treatment” or “treating” of a condition and/or a disease in an individual, including a human or lower mammal, means:
(i) preventing the condition or disease, that is, avoiding any clinical symptoms of the disease, particularly in individuals at risk for developing the condition or disease;
(ii) inhibiting the condition or disease, that is, arresting the development or progression of clinical symptoms; and/or
(iii) relieving the condition or disease, that is, causing the regression of clinical symptoms.

The term “therapeutically effective amount” as defined herein in relation to the treatment of glioblastoma refers to an amount that will decrease, reduce, inhibit, or otherwise abrogate the growth of a glioblastoma cell or tumor. In a specific embodiment, a therapeutically effective amount is an amount that will induce apoptosis in a glioblastoma cell. The specific therapeutically effective amount will vary with such factors as the particular disease being treated, the physical condition of the individual being treated, the duration of the treatment, the nature of concurrent therapy (if any), and the specific formulations employed. In embodiments, the therapeutically effective amount or dose of temozolomide is about 75 mg/m²/day. In other embodiments, the therapeutically effective amount or dosage of temozolomide is about 150-200 mg/m²/day.

The terms “sub-cytotoxic amount, “sub-cytotoxic concentration,” “non-cytotoxic amount,” and “non-cytotoxic concentration,” are used interchangeably herein to refer to an amount or concentration of a drug that is insufficient to kill cancer cells, and more particularly glioblastoma cells. A non-cytotoxic amount of a drug is insufficient, when used alone, to inhibit cancer cell growth or division. According to embodiments of the present disclosure, letrozole is administered at a non-cytotoxic amount or concentration in combination with temozolomide. In some embodiments, a non-cytotoxic dose of letrozole is less than about 1 mg/day, for example, from 0.01 mg/day to 0.9 mg/day. In other embodiments, a non-cytotoxic amount or concentration of letrozole is less than or equal to about 40 nM (0.04 μM), for example, from 0.1 nM to 40 nM.

In embodiments, glioblastoma cells and cell lines may be classified as TMZ-sensitive, TMZ-intermediately sensitive, and TMZ-resistant. In embodiments, a TMZ-sensitive glioblastoma cell or cell line is characterized by inhibition of cell growth with a TMZ IC₅₀ (TMZ concentration where the cell growth is inhibited by 50%) that is below 2 μM in cell culture media. In embodiments, a TMZ-intermediately sensitive glioblastoma cell or cell line is characterized by inhibition of cell growth with a TMZ IC₅₀ that is between 2-20 μM in cell culture media. In embodiments, a TMZ-resistant glioblastoma cell or cell line is characterized by inhibition of cell growth with a TMZ IC₅₀ that is above 20 μM in cell culture media.

As used herein, the terms “administer” or “administration” may comprise administration routes such as enteral (e.g., oral, sublingual, buccal, or rectal), parenteral (e.g., intravenous, intramuscular, subcutaneous, intraarterial, intratumoral), intranasal, inhaled, vaginal, transdermal, etc., so long as the route of administration results in an anti-cancer effect in the subject. In specific embodiments, the administration route is oral, intravenous, or intratumoral. In embodiments, each of temozolomide and letrozole may be administered orally, intravenously, or intratumorally.

“Co-administered,” as used herein, refers to administration of temozolomide and letrozole such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the temozolomide and the letrozole may be administered concurrently or sequentially.

As used herein, the term “subject” generally refers to a living being (e.g., animal or human) capable of suffering from glioblastoma multiforme. In a specific embodiment, the subject is a mammal. In a more specific embodiment, the subject is a human subject.

The present disclosure demonstrates that letrozole synergistically enhances the cytotoxic effects of temozolomide, a DNA alkylating agent that is the first-choice treatment for glioblastoma therapy. Letrozole at nanomolar concentrations markedly potentiated temozolomide activity in patient-derived cell lines having varying sensitivities to temozolomide.

Resistance to temozolomide is a substantial clinical obstacle to the effective treatment of glioblastoma tumors. The magnitude of loss of temozolomide sensitivity is well documented and underscores the clinical failure of temozolomide in the recurrent setting. At doses of 100-200 mg/m² the peak plasma levels of temozolomide are in the range of 5 to 11 μg/ml (25-50 μM). Studies conducted using intracerebral microdialysis to assess the peritumoral temozolomide levels suggest that the partitioning of the drug from plasma to the brain interstitial fluid is only 13-17%, resulting in concentrations ranging from approximately 3-8 μM. With the development of resistance, the tumoral concentrations of TMZ are sub-therapeutic and lead to tumor regression.

Letrozole is an FDA-approved third generation non-steroidal aromatase inhibitor with a sustained record of safe use. The present disclosure further relates to the mechanistic basis for letrozole-mediated potentiation of temozolomide cytotoxicity. While not desiring to be bound by theory, based on the understanding of the function of aromatase as an estrogen synthase in breast cancer tissue, evidence suggests that letrozole treatment results in inhibition of estrogen synthesis, which then triggers a cascade of down-stream effects leading to apoptotic cell death. The apoptotic signaling pathways induced involve down-regulation of Bel-2, up-regulation of Bax, and the activation of caspase-9, caspase-6, and caspase-7. Temozolomide mediates apoptosis by methylating DNA, which primarily occurs at the N⁷ or O⁶ position of guanine residues. This methylation leads to DNA strand breaks and triggers apoptotic tumor cell death.

To understand the synergistic potentiation to temozolomide activity by letrozole, the extent of DNA double strand break was determined by assessing the formation of phosphorylated histone product γH2AX and induction of caspase 3/7 as a marker of apoptosis. γH2AX levels were measured 5 h after drug treatment. No measurable production of γH2AX was observed in TMZ-resistant G75 cells. Letrozole, on the other hand, caused DNA damage in both G75 and G76 cells. Importantly, letrozole at 40 nM concentrations significantly enhanced DNA damage by temozolomide at concentrations as low as 0.1 μM in both cell resistant lines.

Similarly, significant differences were noted in caspase 3/7 induction by temozolomide in G75 (EC₅₀=93.8 μM) and G76 (EC₅₀=6.4 μM) cells, which reduced to 5.1 μM and 1.8 μM, respectively, in the presence of letrozole (40 nM). Concordance was observed for the IC₅₀ values in cell viability assays and the projected EC₅₀ values for caspase 3/7 induction of temozolomide in the presence and the absence of letrozole. While not desiring to be bound by theory, it is believed that the potentiation of temozolomide activity by letrozole is mediated by increased DNA damage resulting in apoptotic cell death in TMZ-sensitive and TMZ-resistant lines.

Again, while not desiring to be bound by theory, the observed effect of letrozole may result, at least partially, from its anti-estrogenic activity. The addition of estradiol to the cell culture media completely abrogated the letrozole effects, as evidenced by protective effects in cell viability and neurosphere growth inhibition assays. Similarly, the presence of estradiol prevented letrozole-mediated DNA damage and induction of apoptosis and negated its synergistic potentiation of temozolomide activity. In the presence of estradiol, the CI analysis plots for cell viability were above the line of additivity, suggesting that that estradiol not only canceled letrozole effects, but may have protected cells from TMZ-mediated damage.

In summary, the present disclosure demonstrates a robust potentiation of temozolomide cytotoxic effects in patient-derived TMZ-sensitive and TMZ-resistant cells by sub-cytotoxic levels of letrozole. Using the quantitative approach of Combination Index analysis, this interaction was shown to be highly synergistic. Letrozole at low and non-cytotoxic concentrations caused substantial reduction in the IC₅₀ values of temozolomide in drug-resistant cells, which suggests that letrozole co-treatment restores temozolomide sensitivity. The present disclosure indicates that combining sub-therapeutic amounts of letrozole with temozolomide robustly increases the anti-tumor efficacy of temozolomide.

In one embodiment, a method of potentiating a cytotoxic effect of temozolomide against glioblastoma cells in a subject in need thereof is provided, the method comprising administering to the subject a combination of: a therapeutically effective amount of temozolomide; and a non-cytotoxic amount of letrozole, wherein the letrozole potentiates the cytotoxic effect of the temozolomide against glioblastoma cells.

In embodiments, the subject is a mammal. In a more specific embodiment, the subject is a human subject.

In embodiments, the compounds of the disclosed methods may be administered via enteral or parenteral routes of administration. In a specific embodiment, enteral administration of temozolomide or letrozole includes oral, sublingual, or buccal administration. In another specific embodiment, parenteral administration of temozolomide or letrozole includes intravenous, intramuscular, subcutaneous, intraarterial, or intratumoral administration. In another specific embodiment, temozolomide and letrozole are each administered orally, intravenously, or intratumorally. In a specific embodiment, temozolomide is administered intravenously or intratumorally and letrozole is administered orally.

Alternatively, treatment with letrozole can precede or follow treatment with temozolomide by intervals ranging from minutes to weeks. Thus, in embodiments, temozolomide and letrozole may be administered concurrently or sequentially. In embodiments where temozolomide and letrozole are applied separately to the cell, one would generally ensure that a significant period of time did not expire between the time of each delivery, such that temozolomide and letrozole would still be able to exert an advantageously combined effect on the cell. In such instances, it is provided that one would contact the cell with both modalities within about 12-24 hours of each other and, optionally, within about 6-12 hours of each other. In some situations, it can be desirable to extend the time period for treatment significantly, however, where several days (2, 3, 4, 5, 6 or 7) to several weeks (1, 2, 3, 4, 5, 6, 7 or 8) lapse between the respective administrations. Also, under some circumstances, more than one administration of either letrozole and/or temozolomide will be desired.

In embodiments of the present methods, temozolomide is administered at a therapeutically effective amount or dose. In a specific embodiment, a therapeutically effective amount of temozolomide is a dose of about 75 mg/m²/day. In another specific embodiment, a therapeutically effective amount of temozolomide is a dose of about 150-200 mg/m²/day.

In embodiments, letrozole is administered at a dose that is non-cytotoxic against glioblastoma cells when administered as a sole therapeutic. In specific embodiments, letrozole is administered at a concentration that is non-cytotoxic against glioblastoma cells that are sensitive, intermediately sensitive, or resistant to temozolomide. In a specific embodiment, a non-cytotoxic amount or dose of letrozole is a dose that is less than 1.0 mg/day. In a more specific embodiment, a non-cytotoxic dose of letrozole ranges from about 0.01 mg/day to about 0.9 mg/day or from about 0.1 mg/day to about 0.9 mg/day. In specific embodiments, the non-cytotoxic dose of letrozole is about 0.01, 0.02, 0.03, 0.04, 0.05, 0.06, 0.07, 0.08, 0.09, 0.1, 0.2, 0.3, 0.4, 0.5, 0.6, 0.7, 0.8, or 0.9 mg/day, or any range therebetween.

In embodiments, a non-cytotoxic concentration of letrozole is less than or equal to about 40 nM (0.04 μM). In specific embodiments, a non-cytotoxic concentration of letrozole ranges from about 0.1 nM to about 40 nM, from about 1 nM to about 40 nM, from about 1 nM to about 30 nM, from about 1 nM to about 20 nM, from about 1 nM to about 10 nM, from about 10 nM to about 40 nM, from about 10 nM to about 30 nM, from about 10 nM to about 20 nM, from about 20 nM to about 40 nM, from about 20 nM to about 30 nM, or from about 30 nM to about 40 nM. In specific embodiments, the non-cytotoxic concentration of letrozole is about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nM, or any range therebetween.

In addition, it will be appreciated that therapeutic benefits can be realized by combining temozolomide and letrozole therapy with one or more additional anti-cancer agents, adjuvants, or treatments. The choice of such combinations will depend on various factors including, but not limited to, the age and general health of the patient, the aggressiveness of disease progression, and the ability of the patient to tolerate the agents that comprise the combination. For example, letrozole and temozolomide can be combined with other agents and therapeutic regimens that are effective at reducing tumor size (e.g., radiation, surgery, chemotherapy, hormonal treatments, and/or gene therapy). In some embodiments, it may be desirable to combine letrozole and temozolomide with one or more agents that treat the side effects of a disease or the side effects of one of the therapeutic agents, e.g., providing the patient with an analgesic, or agents effective to stimulate the patient's own immune response.

A variety of chemical compounds can be used in combination with letrozole and temozolomide. Such compounds include, but are not limited to, chemotherapeutic agents, anti-inflammatory agents, immunosuppressive agents, and corticosteroids. Specific examples of chemotherapeutic agents include, but are not limited to, alkylating agents, platinum drugs, antimetabolites, anti-tumor antibiotics, topoisomerase inhibitors, mitotic inhibitors, targeted therapies, anti-angiogenic compounds, differentiating agents, hormone therapies, and the like.

Suitable alkylating agents include, but are not limited to, (1) nitrogen mustards: such as mechlorethamine, chlorambucil, cyclophosphamide, ifosfamide, and melphalan; (2) nitrosoureas: such as streptozocin, carmustine (BCNU), which may be used for local therapy (for example as Gliadel® wafers), and lomustine; (3) alkyl sulfonates, such as busulfan; (4) triazines, such as dacarbazine (DTIC); and (5) ethylenimines, such as, thiotepa and altretamine (hexamethylmelamine).

Suitable platinum drugs include, but are not limited to, cisplatin, carboplatic, and oxalaplatin.

Suitable antimetabolites include, but are not limited to, 5-fluorouracil (5-FU), 6-mercaptopurine (6-MP), capecitabine, cladribine, clofarabine, cytarabine, floxuridine, fludarabine, gemcitabine, hydroxyurea, methotrexate, premetrexed, pentostatin, and thioguanine.

Suitable anti-tumor antibiotics include, but are not limited to, anthracyclines, such as daunorubicin, doxorubicin, epirubicin, idarubicin, and mitoxantrone; and other anti-tumor antibiotics such as actinomycin-D, bleomycin, and mitomycin-C.

Suitable topoisomerase inhibitors include, but are not limited to, topotecan and irinotecan (CPT-11) and topoisomerase II inhibitors such as etoposide (VP-16), teniposide, and mitoxantrone.

Suitable mitotic inhibitors include, but are not limited to, taxanes, such as paclitaxel (also referred to as taxol) and docetaxel; epothilones such as ixabepilone; vinca alkaloids such as vinblastine, vincristine, vinorelbine, and estramustine.

Suitable targeted therapies include, but are not limited to, (1) small molecules, such as imatinib, gefitinib, nilotinib, lapatinib, sunitinib, and axitinib; (2) monoclonal antibodies, such as bevacizumab (Avastin®), alemtuzumab, cetuximab, rituximab, and trastuzumab; (3) PI3 kinase inhibitors, such as BEA235; and (4) inhibitors of the mammalian target of rapamycin (mTOR), such as everolimus, sirolimus, and tacrolimus.

Anti-angiogenic compounds suitable for combination with letrozole and temozolomide include, but are not limited to, axitinib, and bevacizumab (Avastin®).

Suitable differentiating agents include, but are not limited to, retinoids, tretinoin (ATRA or Atralin®), bexarotene (Targretin®), and arsenic trioxide (Arsenox®).

Hormone therapies suitable for combination with letrozole and temozolomide include, but are not limited to, anti-estrogens such as tamoxifen, toremifene, and fulvestrant.

Additional chemotherapeutic agents suitable for combination with letrozole and temozolomide include L-asparaginase and the proteosome inhibitor bortezomib.

Suitable corticosteroids include, but are not limited to, prednisone, methylprednisolone (Solumedrol®), and dexamethasone (Decadron®).

Letrozole and temozolomide combination therapy may also be combined with certain devices or alternative therapies. For example, the combination therapy of letrozole and temozolomide may be combined with therapies that employ electric fields to disrupt cell division (such as tumor treating fields (TTF) therapy by Novocure™). In another embodiment, letrozole and temozolomide may be combined with boron neutron capture therapy.

Combination treatments involving letrozole, temozolomide, and another therapeutic agent can be achieved by co-administering the agents, i.e., contacting cells with all agents at the same time. Such combinations can be achieved by contacting the cell with a single composition or pharmaceutical formulation that includes all agents, or by contacting the cell with distinct compositions or formulations, at the same time, wherein one composition includes letrozole, one composition comprises letrozole, and one composition includes the additional anti-cancer agent.

Alternatively, treatment with letrozole and temozolomide can precede or follow treatment with the additional anti-cancer agent by intervals ranging from minutes to weeks. In embodiments where the additional agent and letrozole and temozolomide are applied separately to the cell, one would generally ensure that a significant period of time did not expire between the time of each delivery, such that the agent, letrozole, and temozolomide would still be able to exert an advantageously combined effect on the cell. In such instances, it is provided that one would contact the cell with all modalities within about 12-24 hours of each other and, optionally, within about 6-12 hours of each other. In some situations, it can be desirable to extend the time period for treatment significantly, however, where several days (2, 3, 4, 5, 6 or 7) to several weeks (1, 2, 3, 4, 5, 6, 7 or 8) lapse between the respective administrations. Also, under some circumstances, more than one administration of either letrozole, temozolomide, and/or the other agent will be desired.

Additional cancer treatments also can be used in combination with administration letrozole and temozolomide. For example, letrozole and temozolomide can be used as part of a treatment course further involving attempts to surgically remove part or all of a cancerous growth. For instance, letrozole and temozolomide can be administered after surgical treatment of a patient to treat any remaining cancer cells. Treatment with letrozole and temozolomide can precede surgery, in an effort to shrink the size of a tumor to reduce the amount of tissue to be excised, thereby making the surgery less invasive and traumatic. Letrozole and temozolomide can also be administered during surgery, as a means of regionally administering the drug to the treatment area.

Treating glioblastoma tumors with letrozole and temozolomide as described herein can further include one or more treatment courses with a radiotherapeutic agent to induce DNA damage. Radiotherapeutic agents include, for example, gamma irradiation, X-rays, UV-irradiation, microwaves, electronic emissions, radioisotopes, and the like. Therapy can be achieved by irradiating the localized tumor site with the above-described forms of radiation.

In embodiments, the one or more additional active agents are selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, and a hormone therapy.

In embodiments of the present methods, the glioblastoma cells treated with temozolomide and letrozole are TMZ-sensitive, TMZ-intermediately sensitive, or TMZ-resistant cells. In a specific embodiment, the glioblastoma cells are TMZ-resistant cells.

In another embodiment, a method of restoring sensitivity of temozolomide-resistant glioblastoma cells to temozolomide is provided, the method comprising contacting the glioblastoma cells with a non-cytotoxic amount of letrozole as described herein. In a specific embodiment, the non-cytotoxic amount of letrozole is selected from a dose less than 1.0 mg/day (for example, from 0.01 mg/day to 0.9 mg/day) or a concentration of about 40 nM or less (for example, from 0.1 nM to 40 nM).

In another embodiment, a combination of a therapeutically effective amount of temozolomide and a non-cytotoxic amount of letrozole is provided for use in a method of treating glioblastoma in a subject in need thereof. In a specific embodiment, the non-cytotoxic amount of letrozole is a dose less than 1.0 mg/day (for example, from 0.01 mg/day to 0.9 mg/day). In another specific embodiment, the non-cytotoxic amount of letrozole is a concentration of about 40 nM or less (for example, from 0.1 nM to 40 nM). Optionally, the glioblastoma cells are TMZ-sensitive, TMZ-intermediately sensitive, or TMZ-resistant cells.

In another embodiment, a method of treating glioblastoma in a subject in need thereof is provided, the method comprising administering to the subject a combination of: a therapeutically effective amount of temozolomide; and a non-cytotoxic amount of letrozole. In a specific embodiment, the non-cytotoxic amount of letrozole is a dose less than 1.0 mg/day (for example, from 0.01 mg/day to 0.9 mg/day). In another specific embodiment, the non-cytotoxic amount of letrozole is a concentration of about 40 nM or less (for example, from 0.1 nM to 40 nM). In embodiments, the glioblastoma is TMZ-sensitive, TMZ-intermediately sensitive, or TMZ-resistant glioblastoma.

### EXAMPLES

The following examples are given by way of illustration are not intended to limit the scope of the disclosure.

### Example 1. Materials and Methods

Materials: Patient-derived GBM cells G43, G75, and G76 were kindly provided by Dr. Jann Sarkaria, (Mayo Clinic Brain Tumor Patient-Derived Xenograft National Resource). BT-142 gfp luc cells were obtained from Cincinnati Children's Hospital Medical Center. Neural stem cell culture medium, Gibco™ StemPro™ NSC SFM (Catalog #A1050901), penicillin (50 U/mL), streptomycin (50 mg/mL), letrozole (99% pure), and CyQuant™ LDH assay kit were purchased from Fisher Scientific. Temozolomide was purchased from Toronto Research Chemicals Inc. Enzyme linked immunoassay kit for estradiol was purchased from Fisher Scientific (Invitrogen Human Estradiol ELISA Kit REF #: KAQ0621). Caspase-Glo 3/7® Assay kit was purchased from Promega Inc. Rabbit monoclonal anti-gamma H2A.X (phospho S139) antibody [9F3](ab26350) was obtained from Abcam Inc.

Cell culture and maintenance: Patient-derived GBM cells were grown and maintained in neural stem cell growth medium (StemPro™ NSC SFM; Gibco™ Cat #A1050901) supplemented with NSC growth supplement, hEFG, hFGF and 10% penicillin and streptomycin. Serum-free medium enables spheroid formation in the patient-derived cells; hence use of any serum rich material was avoided. Cells were cultured in T-25 flasks until 70-80% confluence was reached, at which point the spheroids formed were disrupted into single cells using trituration with a P200 pipette. The confluent single cell suspensions were used for cell counting using a hemocytometer and trypan blue exclusion and seeded into appropriate plates at appropriate densities for further assays.

Cell viability Assay: The cytotoxic effects of TMZ, LTZ, and various combinations of the two agents were evaluated using the CyQuant™ Lactate Dehydrogenase (LDH) assay (ThermoFisher Scientific). This assay measures the release of LDH, a cytosolic enzyme, into the extracellular medium resulting from damage to the plasma membrane. Cells (1.4×10⁴) were plated in 96 well plates in serum-free medium. Twenty-four hours after seeding, cells were treated with TMZ (0-100 μM), LTZ (0-2 μM), or TMZ (0-10 μM)+LTZ (0.04 μM). Dimethyl sulfoxide (0.1%) was used as a vehicle control. The effect of adding estradiol (250 pg/ml) on cell viability was also determined.

Neurosphere Growth Inhibition: Since G43 and BT-142 cells form neurospheres in culture medium, the effects of LTZ and TMZ on the growth of the neurosphere. Cells (1×10⁴) were plated and 24 hours later treated with LTZ (0-2 μM), TMZ (0-100 μM) or were also determined. Cells were treated with LTZ (0.04 μM)+TMZ (0-10 μM) for 72 h cells, with DMSO (0.1%) serving as vehicle control. Neurosphere dimensions were measured using Leica Stereomicroscope in bright field setting. The effect of addition of estradiol (250 pg/ml) on the neurosphere growth was also determined.

Combination Index Analysis: Combination Index (CI) analyses were employed to evaluate the nature of the interaction between LTZ and TMZ. For each cell-line, 1.4×10⁴ cells were seeded in each well of a 96-well plate and allowed to grow for 24 h. Cells were treated with LTZ, TMZ, and LTZ+TMZ (0-10 μM) at a fixed LTZ:TMZ ratio across the concentration range. The cytotoxicity of the combination was determined using the LDH assay and the fraction affected (% cell kill relative to control) at each combination was plotted using the Compusyn® software. For CI analysis of the neurosphere growth inhibition by LTZ and TMZ, 1.4×10⁴ BT-142-gfp-luc and G43 cells were plated per well of a 96-well plate and allowed to grow for 24 h. Cells were treated with LTZ, TMZ and LTZ+TMZ (0-10 μM) at a fixed LTZ:TMZ ratio across the concentration range. Microscopic images were captured 72 h after the treatment and the fraction affected (% decrease in neurosphere size relative to control) at each concentration of the combination was plotted using Compusyn® Software. The CI values were determined using the following equation: CI=(C)1/(Cx)1+(C)2/(Cx)2, where (Cx)1, (Cx)2=the concentration of the tested substance 1 and the tested substance 2 used in the single treatment that was required to decrease the cell number by x % and (C)1, (C)2=the concentration of the tested substance 1 in combination with the concentration of the tested substance 2 that together decreased the cell number by x %. The CI value quantitatively defines synergism (CI<1), additive effect (CI=1) and antagonism (CI>1). These experiments were repeated in the presence of estradiol to assess the extent to which estradiol impacts the cytotoxic effects of the combination.

Assessment of gamma-H2A.X: Induction of γH2A.X in G75 and G76 cells (lx 106) treated with LTZ, TMZ, and LTZ+TMZ (0-10 μM) in the presence and the absence of estradiol (250 pg/ml) was assessed using flow cytometric analysis. Based on initial assessment of the time course of γH2A.X induction, a 5 h treatment was used to capture maximal effect. Cell pellets were collected by centrifugation of the cells at 2,000×g for 5 minutes at room temperature. Cells were then washed with ice cold PBS, followed by addition of a 1:1 mixture of methanol and NP40 (300 μL) for fixation and permeabilization of the cells. Following centrifugation at 4° C. at 290×g, 100 μL of 1:250 dilution of the primary unconjugated antibody for gamma-H2A.X at room temperature were added to the cells. The cells were incubated with the primary antibody for 20 min and washed with 600 μL of 1:1 methanol and NP40 mixture. Secondary Per-CP conjugated antibody was added to the cells at a dilution of 1:1000 and incubated at room temperature in the dark for 20 minutes. Cells were washed again with 600 μL of 1:1 methanol and NP40 mixture, re-suspended in 1×PBS, and subjected to flow cytometric analysis with the excitation wavelength of 488 nm (Beckman CytoFlex flow cytometer). Gating was done using human IgG-isotype control as suggested by the PE antibody manufacturer. The scatter plots were analyzed using CytExpert version 2.3 to assess the percentage of cells positive for γH2A.X versus the isotype control.

Apoptosis induction assay using Caspase-Glo® 3/7 system: G75 and G76 cells were employed to assess the effects of LTZ, TMZ, and LTZ+TMZ combination treatment on induction of apoptosis through caspase 3/7 activation. Cells were seeded at a density of 1.5×10⁴ cells per well in a translucent walled, clear bottom, 96-well plate. Promega Caspase-Glo® 3/7 Assay system was used to assess the caspase 3/7 levels in cells treated with LTZ, TMZ, LTZ (0.04 μM)+TMZ (0-5 μM), and vehicle (0.1% DMSO). Cells were treated for 24 h after plating. Caspase-Glo® 3/7 3D substrate and Caspase-Glo® 3/7 3D buffer was mixed to form a 10 mL reaction mixture and 100 μL of the mixture was added to each well. The reaction was allowed to proceed at room temperature in the dark for 30 minutes. The luminescence readings were carried out 24 h after the treatment using Synergy™ HTX Multi-Mode Microplate Reader. The experiment was repeated in presence of estradiol to assess the effect of estradiol on caspase induction in G75 and G76 cells.

Statistical Analysis: The results were expressed as mean values±standard deviation (SD). Statistical comparisons were analyzed by one-way analysis of variance (ANOVA) using GraphPad Prism 8. A significance level of P<0.05 was used for all tests.

### Example 2. Potentiation of Cytotoxicity and Neurosphere Growth Inhibition of TMZ by LTZ

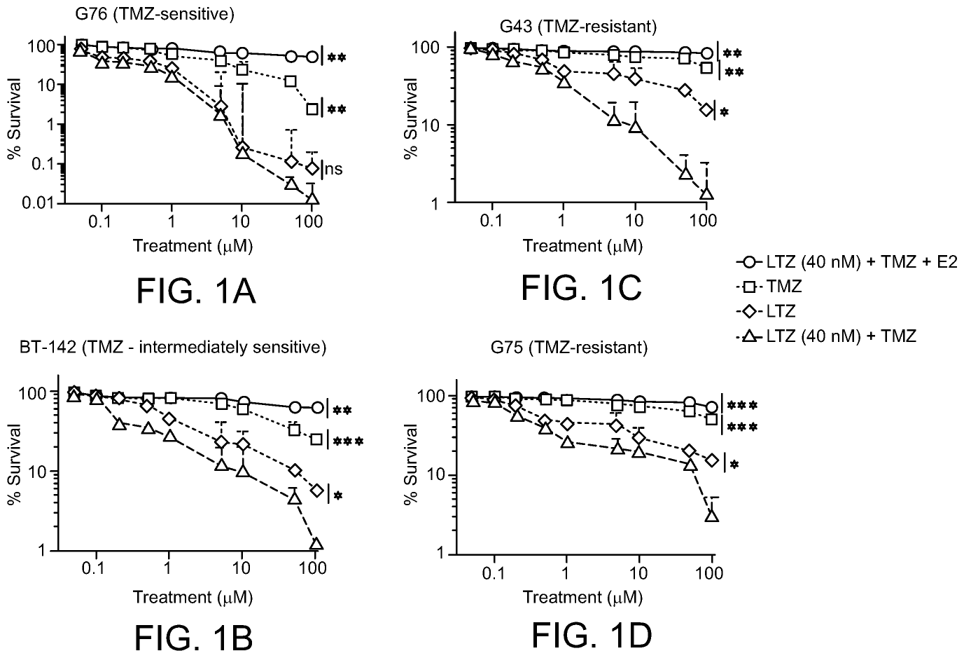

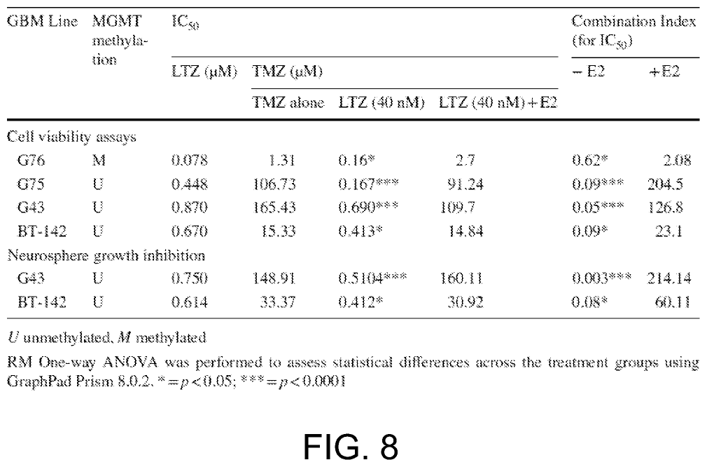

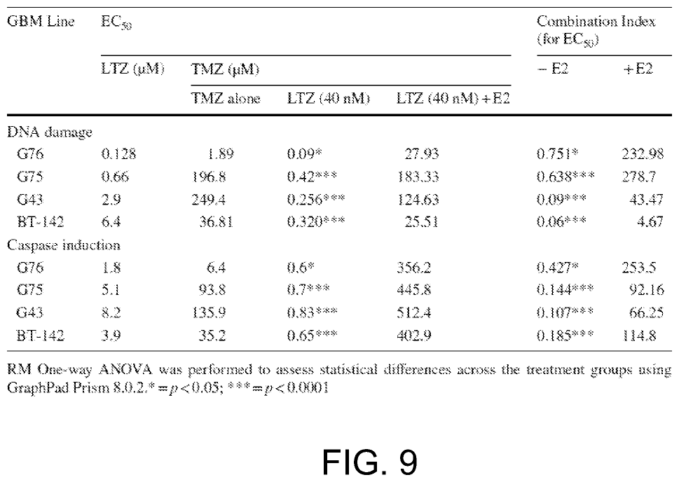

The cytotoxic effects of TMZ, LTZ, and TMZ+LTZ (40 nM) on various patient-derived GBM lines were examined. Cells were treated for 72 h and the IC₅₀ values for LTZ and TMZ cytotoxicity were determined. As shown in FIGS. 1A-1D, the TMZ IC₅₀ values ranged from 1.3 μM (G76) to 165 μM (G43). As evidenced by this initial cell viability assessment, G76 cells are TMZ-sensitive (IC₅₀=1.31 μM), BT-142 cells have intermediate TMZ sensitivity (IC₅₀=15.33 μM), and G75 and G43 cells are clearly TMZ resistant, having TMZ IC₅₀ values of 106.73 and 165.43 μM, respectively (see FIG. 8). The cells were fairly sensitive to LTZ, with IC₅₀ values ranging from 0.08 to 0.67 μM. Combining LTZ at a non-cytotoxic concentration of 40 nM with TMZ strikingly enhanced TMZ cytotoxicity. As shown in the table of FIG. 1, the TMZ IC₅₀ values in the presence of LTZ (40 nM) ranged from 0.16 to 0.69 μM for all four cell lines.

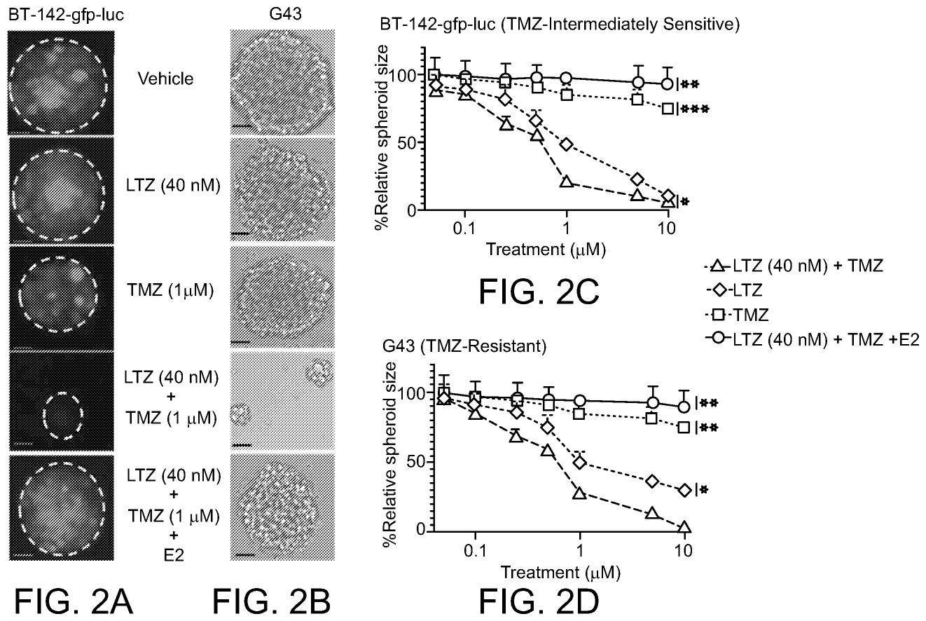

The potentiation of TMZ cytotoxicity in the presence of LTZ was also evident in neurosphere growth assay. TMZ-sensitive BT-142 and TMZ-resistant G43 cells were treated for 72 h and neurospheres were visualized via microscopy. FIGS. 2A-2B provide representative images of neurospheres treated with solvent, LTZ (40 nM), TMZ (1 μM), and TMZ (1 μM)±LTZ (40 nM). In BT-142 cells, the median neurosphere diameter in wells treated with the solvent vehicle, LTZ (40 nM), TMZ (1 μM), or TMZ (1 μM)±LTZ (40 nM) were, respectively, 1.3±0.09, 1.1±0.077, 0.9±0.023, and 0.23±0.05 mm. Likewise, in G43 cells the neurosphere diameters measured in wells treated with the solvent vehicle, LTZ (40 nM), TMZ (1 μM), or TMZ (1 μM)±LTZ (40 nM) were, respectively, 1.9±0.11, 1.85±0.13, 1.6±0.085 and 0.05±0.01.

As shown in FIGS. 2C-2D, while LTZ (40 nM) minimally impacted neurosphere growth, when combined with TMZ, the combination markedly enhanced neurosphere growth inhibition by TMZ in both tested cell lines.

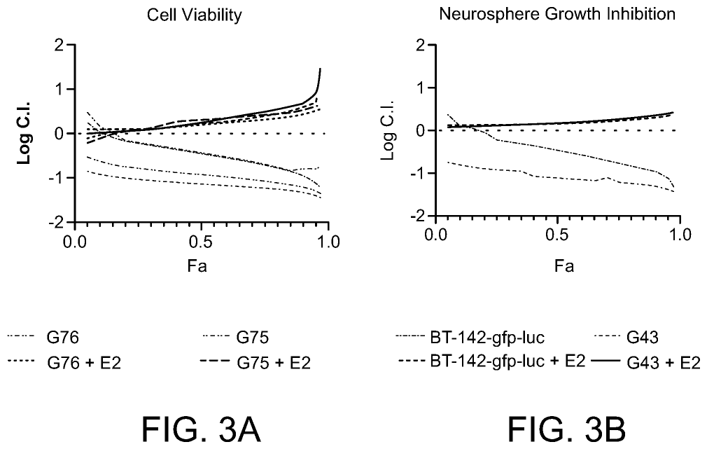

Combination index analysis of cell viability and neurosphere growth inhibition was carried out to determine whether the observed potentiation of TMZ activity was additive or synergistic. The combination index (CI) plots are set forth in FIGS. 3A-3B. In each case, the CI values were consistently well below the line of additivity (CI=1), indicative of strongly synergistic interaction between LTZ and TMZ. CI values at the IC₅₀ values ranged from 0.05 and 0.09 for TMZ-resistant G43 and G75 cells, which is consistent with the striking reduction in TMZ IC₅₀ values. The extent of synergy was considerably greater for the TMZ-resistant G43 and G75 lines, which is consistent with the high IC₅₀ values of TMZ against these lines; as such, the fold decrease in IC₅₀ values is much higher for these cell lines compared to the TMZ-sensitive cell lines.

### Example 3. Assessment of DNA Damage and Apoptosis by TMZ and LTZ

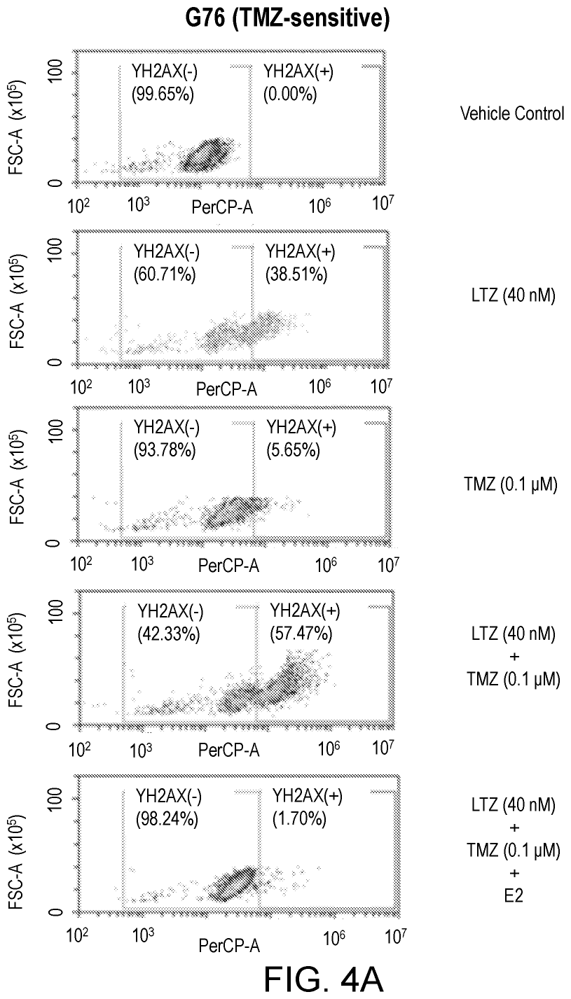

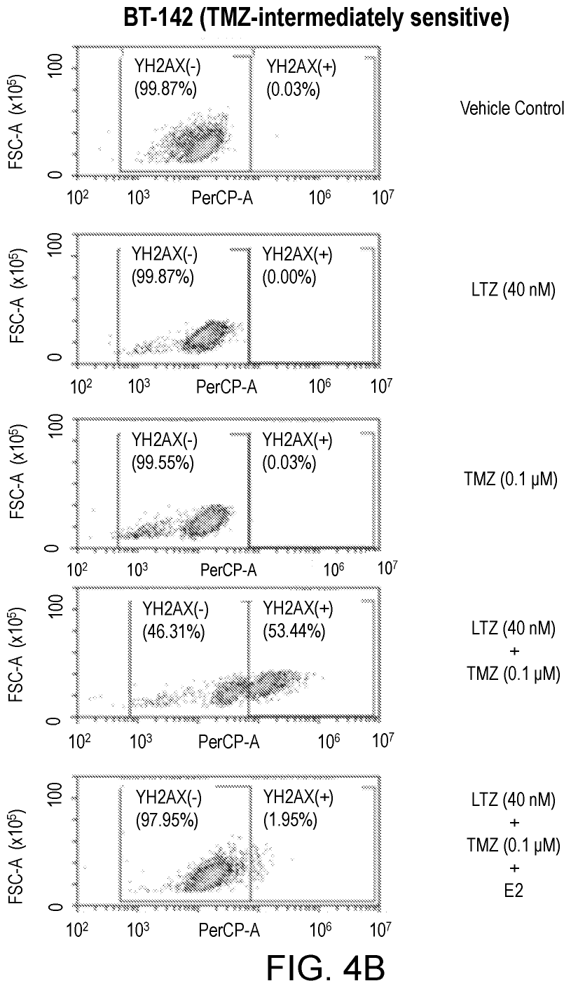

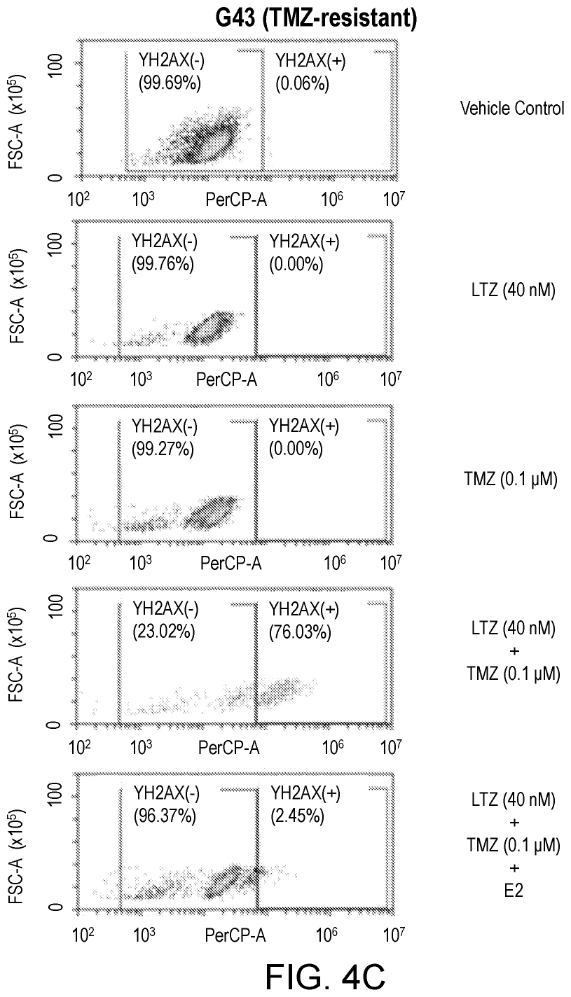

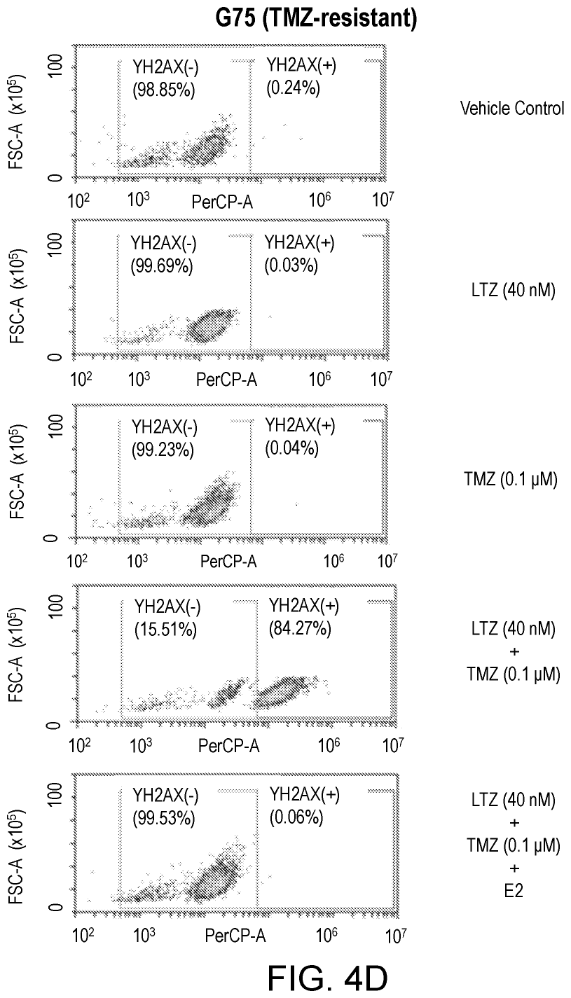

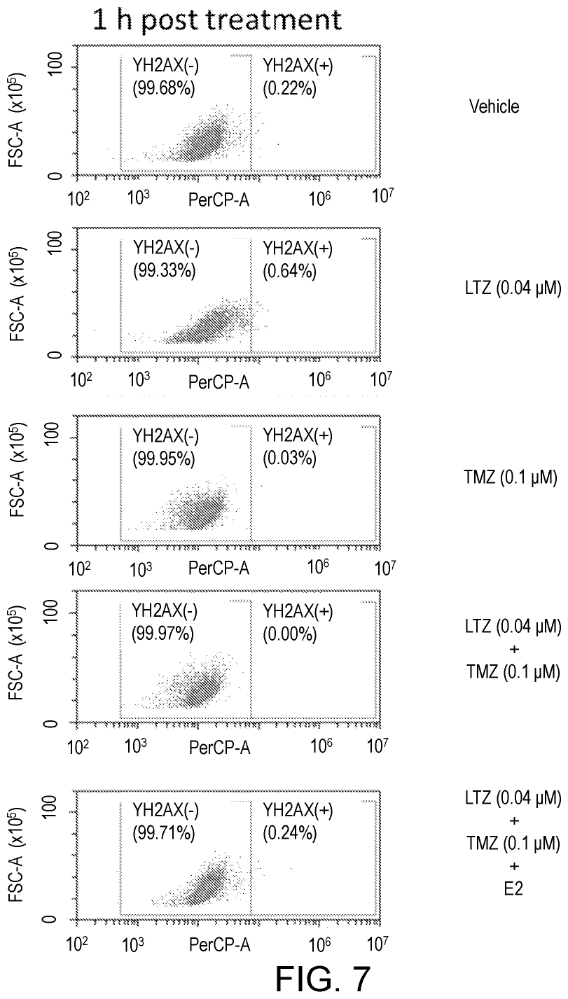

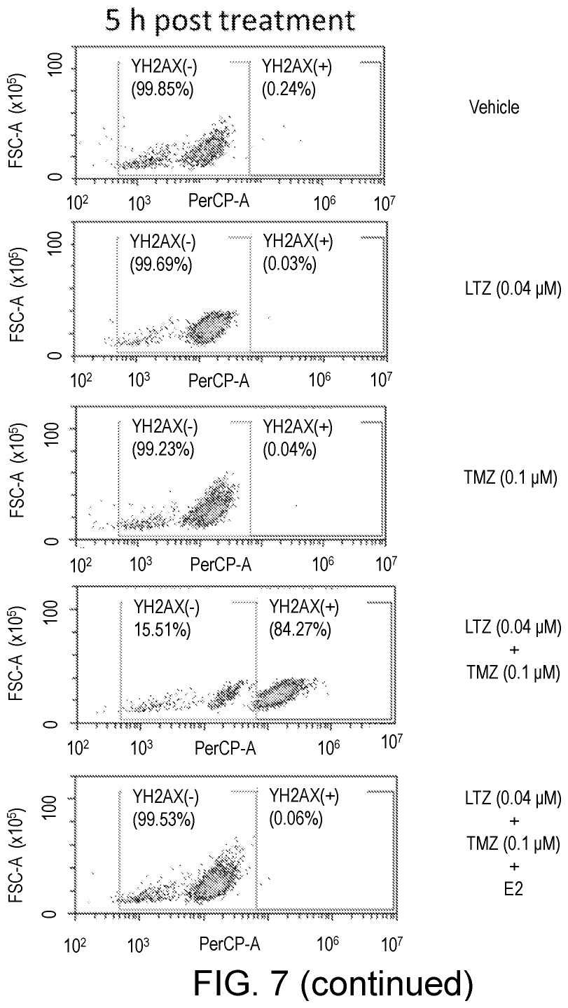

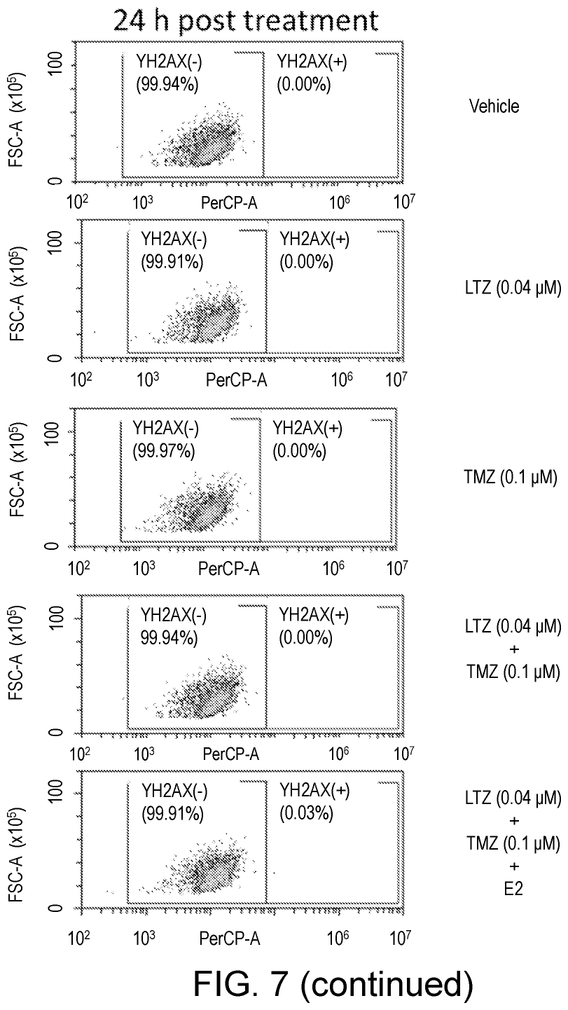

The mechanism of cell death by DNA alkylating agents such as TMZ entails increased DNA damage leading to double strand DNA breaks and subsequent tumor cell apoptosis. Accordingly, the DNA damaging and apoptotic effects of TMZ+LTZ (40 nM) on TMZ-sensitive (G76) and TMZ-resistant cell (G75) lines were assessed. The phosphorylation of the histone H2AX following double strand DNA breaks to form γ-H2AX was assessed as a marker for DNA damage. The detection and visualization of γ-H2AX was carried out via flow cytometry. Initial optimization of the method revealed that maximal γ-H2AX levels were detected approximately 5 h following drug treatment (see FIG. 7). As such, γ-H2AX formation in G75 and G76 cells was measured following a 5 h treatment with TMZ and LTZ combinations. The effect of LTZ (40 nM) and the addition of estradiol to the cell culture medium was also investigated. As shown in FIGS. 4A-4D, TMZ at concentrations up to 1 μM had noticeable induction of γ-H2AX in G76 cells, but not in G75 cells. On the other hand, LTZ (500 nM and 1 μM) considerably increased γ-H2AX levels. Moreover, LTZ (40 nM) when combined with TMZ (0.1 μM) had a dramatic impact on the number of cells that stained positive for γ-H2AX levels. The effects of LTZ-mediated induction of γ-H2AX and were abrogated in the presence of estradiol.

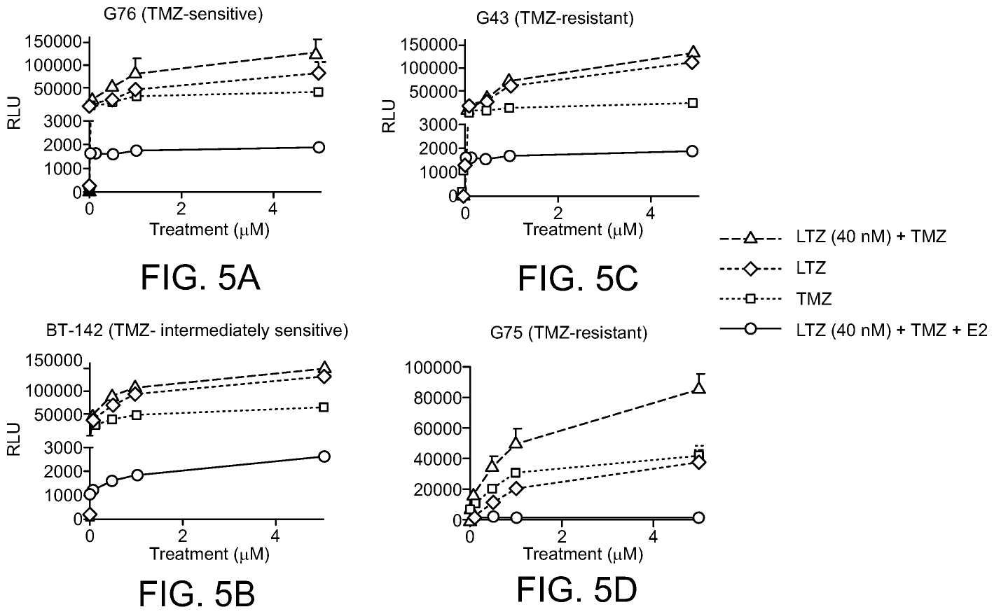

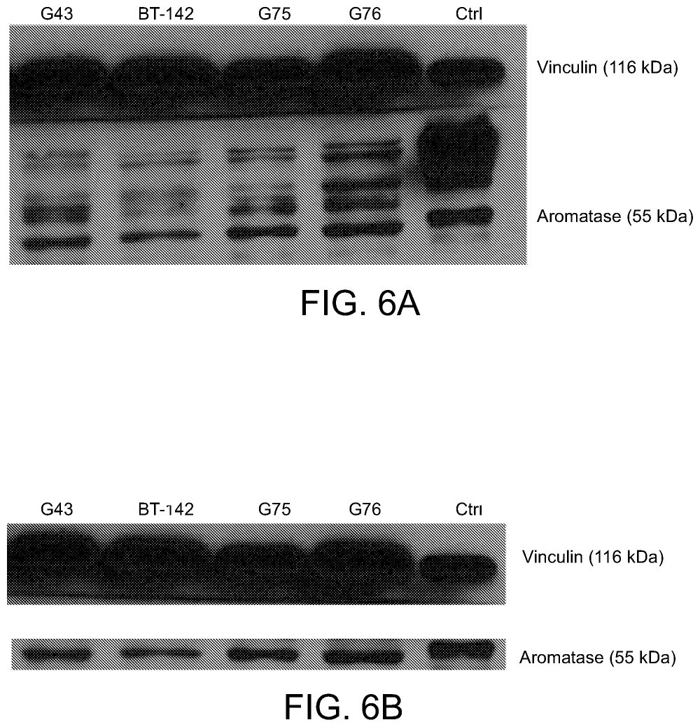

Caspase-3/caspase-7 activity was assayed as a marked of apoptosis in cells treated with TMZ and LTZ. Caspase 3 and caspase 7 are cysteine-aspartic acid proteases which can directly execute apoptosis following sequential activation of caspase-8 or caspase-9. As such, induction of caspase 3/caspase 7 is often used to measure cellular apoptosis. The Caspase-Glo® 3/7 luminescence assay was used to determine induction of apoptosis following 72 h drug exposure in G75 and G76 cells. As shown in FIGS. 5A-5D, when employed at concentrations up to 5 μM, TMZ induced apoptosis in G76 cells, but not in G75 cells. LTZ, on the other hand, was very effective in inducing apoptosis in a concentration-dependent manner with EC₅₀ values of 1.8±0.312 and 5.07±1.102 μM. Importantly, LTZ (40 nM) caused a striking increase in TMZ-mediated apoptosis in both G75 and G76 cells (see FIGS. 5A, 5D).

Aspects of the present disclosure can be described with reference to the following numbered clauses, with preferred features laid out in dependent clauses.
1. A method of potentiating a cytotoxic effect of temozolomide against glioblastoma cells in a subject in need thereof, the method comprising administering to the subject a combination of:
   a therapeutically effective amount of temozolomide; and
   a non-cytotoxic amount of letrozole,
   wherein the letrozole potentiates the cytotoxic effect of the temozolomide.
2. The method according to clause 1, wherein the subject is a mammal.
3. The method according to clause 1 or clause 2, wherein the subject is a human.
4. The method according to any of the preceding clauses, wherein administering comprises enteral or parenteral administration.
5. The method according to clause 4, wherein enteral administration comprises oral, sublingual, or buccal administration.
6. The method according to clause 4, wherein parenteral administration comprises intravenous, intramuscular, subcutaneous, intraarterial, or intratumoral administration.
7. The method according to any of the preceding clauses, wherein the temozolomide is administered at a dose of about 75 mg/m²/day.
8. The method according to any of clauses 1-7, wherein the letrozole is administered at a dose of from about 0.01 mg/day to 0.9 mg/day.
9. The method according to any of clauses 1-7, wherein the letrozole is administered at a concentration of from about 0.1 nM to 40 nM.
10. The method according to any of the preceding clauses, wherein the temozolomide is administered orally, intravenously, or intratumorally and the letrozole is administered orally.
11. The method according to any of the preceding clauses, wherein the temozolomide and the letrozole are administered concurrently or sequentially.
12. The method according to any of the preceding clauses, further comprising administering to the subject one or more additional active agents selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, and a hormone therapy.
13. The method according to clause 12, wherein the chemotherapeutic agent is bevacizumab or carmustine.
14. The method according to any of the preceding clauses, further comprising administering radiation therapy to the subject.
15. The method according to any of the preceding claims, wherein the glioblastoma cells are TMZ-sensitive, TMZ-intermediately sensitive, or TMZ-resistant cells.
16. The method according to any of the preceding clauses, wherein the temozolomide and the letrozole induce apoptosis in glioblastoma cells.
17. A method of restoring sensitivity of temozolomide-resistant glioblastoma cells to temozolomide, the method comprising contacting the glioblastoma cells with a non-cytotoxic amount of letrozole.
18. The method according to clause 17, wherein the non-cytotoxic amount of letrozole is a dose of from about 0.01 mg/day to 0.9 mg/day; or a concentration of from about 0.1 nM to 40 nM.
19. A combination of a therapeutically effective amount of temozolomide and a non-cytotoxic amount of letrozole for use in a method of treating glioblastoma in a subject in need thereof.
20. The combination for use according to clause 19, wherein the non-cytotoxic amount of letrozole is a dose of from about 0.01 mg/day to 0.9 mg/day.
21. The combination for use according to clause 19 or clause 20, wherein the non-cytotoxic amount of letrozole is a concentration of from about 0.1 nM to 40 nM.
22. The combination for use according to any of clauses 19-21, wherein the letrozole potentiates an apoptotic-inducing effect of temozolomide against glioblastoma cells.
23. The combination for use according to any of clauses 19-22, wherein the glioblastoma cells are TMZ-sensitive, TMZ-intermediately sensitive, or TMZ-resistant cells.
24. The combination for use according to any of clauses 19-23, wherein the subject is a mammal, preferably a human.
25. The combination for use according to any of clauses 19-24, wherein the temozolomide and the letrozole are administered to the subject enterally or parenterally.
26. The combination for use according to any of clauses 19-25, wherein the temozolomide and the letrozole are administered concurrently or sequentially.
27. A method of treating glioblastoma in a subject in need thereof, the method comprising administering to the subject a combination of:
   a therapeutically effective amount of temozolomide; and
   a non-cytotoxic amount of letrozole.
28. The method according to clause 27, wherein the glioblastoma is TMZ-sensitive, TMZ-intermediately sensitive, or TMZ-resistant glioblastoma.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method of potentiating a cytotoxic effect of temozolomide against glioblastoma cells in a subject in need thereof, the method comprising administering to the subject a combination of:
a therapeutically effective amount of temozolomide; and
a non-cytotoxic amount of letrozole,
wherein the letrozole potentiates the cytotoxic effect of the temozolomide against the glioblastoma cells.

2. The method according to claim 1, wherein the subject is a mammal.

3. The method according to claim 2, wherein the subject is a human.

4. The method according to claim 1, wherein administering comprises enteral or parenteral administration.

5. The method according to claim 4, wherein enteral administration comprises oral, sublingual, or buccal administration.

6. The method according to claim 4, wherein parenteral administration comprises intravenous, intramuscular, subcutaneous, intraarterial, or intratumoral administration.

7. The method according to claim 1, wherein the temozolomide is administered at a dose of about 75 mg/m²/day.

8. The method according to claim 1, wherein the letrozole is administered at a dose of from about 0.01 mg/day to 0.9 mg/day.

9. The method according to claim 1, wherein the letrozole is administered at a concentration of from about 0.1 nM to 40 nM.

10. The method according to claim 1, wherein the temozolomide is administered orally, intravenously, or intratumorally and the letrozole is administered orally.

11. The method according to claim 1, wherein the temozolomide and the letrozole are administered concurrently or sequentially.

12. The method according to claim 1, further comprising administering to the subject one or more additional active agents selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, and a hormone therapy.

13. The method according to claim 12, wherein the chemotherapeutic agent is bevacizumab or carmustine.

14. The method according to claim 1, further comprising administering radiation therapy to the subject.

15. The method according to claim 1, wherein the glioblastoma cells are TMZ-sensitive, TMZ-intermediately sensitive, or TMZ-resistant cells.

16. The method according to claim 1, wherein the temozolomide and the letrozole induce apoptosis in glioblastoma cells.

17. A method of restoring sensitivity of temozolomide-resistant glioblastoma cells to temozolomide, the method comprising contacting the glioblastoma cells with a non-cytotoxic amount of letrozole.

18. The method according to claim 17, wherein the non-cytotoxic amount of letrozole is a dose of from about 0.01 mg/day to 0.9 mg/day; or a concentration of from about 0.1 nM to 40 nM.

19.-26. (canceled)

27. A method of treating glioblastoma in a subject in need thereof, the method comprising administering to the subject a combination of:
a therapeutically effective amount of temozolomide; and
a non-cytotoxic amount of letrozole.

28. The method according to claim 27, wherein the glioblastoma is TMZ-sensitive, TMZ-intermediately sensitive, or TMZ-resistant glioblastoma.
